(19) United States
(12) Patent Application Publication
(10) Pub. No.: US 2005/0143714 A1
(43) Pub. Date: Jun. 30, 2005

(54) Sutureless pump connector

(75) Inventors: Michael Hegland, Mounds View, MN (US); James M. Olsen, Plymouth, MN (US); James G. Skakoon, St. Paul, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/950,727

(22) Filed: Sep. 27, 2004

Related U.S. Application Data
(60) Provisional application No. 60/506,594, filed on Sep. 26, 2003.

(51) Int. Cl. A61M 25/16

(52) U.S. Cl. 604/533

(57) ABSTRACT

Sutureless pump connectors are discussed. The sutureless pump connectors include a restraint and a lock. The restraint has a proximal portion capable of engaging a portion of an implantable pump, such as a port. The restraint also has a distal portion capable of engaging a catheter. The lock is configured to secure the restraint to secured position relative to the pump and the catheter. The restraint may have a tab configured to secure the lock on the restraint. The tabs may be configured to releasably secure the lock.

### RELATED APPLICATION

This application claims priority to provisional U.S. Application Ser. No. 60/506,594, filed Sep. 26, 2003.

### FIELD

This application relates to connectors for connecting medical devices.

### BACKGROUND

Implantable infusion pump systems may be a preferred delivery system when it is important to deliver a therapeutic agent to a specific site or when the agent must be administered to sites in tightly controlled, yet minute dosages. Such pump systems typically contain a reservoir operably connected to a catheter. The reservoir, which holds the therapeutic agent, may be disposed within the housing of the infusion pump. The catheter is typically connected to the pump by sutures. With sutures, the catheter can disconnect from the pump between about 0.7% and about 1.4% of the time in clinical use. In addition, undesired cuts and punctures are a source of complications with catheters sutured to pumps, occurring at a rate of about 0.6% to about 6.2% of the time in clinical use. Further, use of sutures to secure catheters to pumps is a source of variability and is subject to variability in tightness. Replacement of a catheter secured by sutures may also be a source of concern, as caution is needed in removing a suture from a pump connector.

### BRIEF SUMMARY

An embodiment of the invention provides a connector for securing a catheter to an implantable pump. The connector comprises a restraint configured to secure an implantable pump relative to a catheter. The connector further comprises a lock configured to secure the restraint in a secured position relative to the pump and the catheter. The restraint may be configured to engage the pump upon compression, in a radially inward direction, of at least a portion of the restraint. The lock may provide the compression. Alternatively, the restraint may be configured to engage the pump in a relaxed state.

An embodiment of the invention provides a catheter comprising a connector. The catheter comprises a tubular member having a distal portion and a proximal portion. The proximal portion of the tubular member is configured for connection to an implantable infusion pump. The catheter may further comprise a seal disposed about the proximal portion of the tubular member. The catheter or seal may comprise an element. The catheter further comprises a restraint disposed about the catheter or seal. The restraint is configured to engage a portion of the implantable infusion pump o secure the catheter to the pump. The restraint may comprise a mating element configured to engage the element of the catheter or seal. The restraint mating element is complementary to the element of the catheter or seal.

An embodiment of the invention provides a system comprising an implantable infusion pump, a catheter, and a connector configured to secure the catheter relative to the pump. The connector comprises a restraint configured to secure the proximal end of the catheter to the implantable pump. The connector further comprises a lock configured to secure the restraint in a longitudinal position relative to the pump and the catheter.

At least one or more of the connectors, catheters, systems, and methods described herein may provide an advantage over currently existing pump connector technology. For example, the pump connectors as described herein may connect a catheter to a pump thereby reducing implant procedure time or minimizing impact of suture technique variability. The connectors may also reduce risks associated with implantation techniques requiring sutures. Further, the ease of reversibility of a connection between a catheter and a pump by using a connector as described herein may prove advantageous. These and other advantages will become evident to one skilled in the art upon reading the disclosure herein.

### BRIEF DESCRIPTION OF THE DRAWINGS

The advantages of the present invention will become more apparent by referring to the following detailed description and accompanying drawings, in which:

FIG. 1 is a drawing showing an embodiment of the invention where a catheter is connected to a pump by a sliding ring type connector;

FIG. 2 is a drawing showing an embodiment of the invention where a catheter is connected to a pump by a snap type connector;

FIG. 3 is a drawing showing an embodiment of the invention where a catheter is connected to a pump by a snap rings type connector;

FIG. 4 is a drawing showing an embodiment of the invention where a catheter is connected to a pump by a sliding ring type connector;

FIG. 5 is a drawing showing an embodiment of the invention where a catheter is connected to a pump by a screw on ring type connector;

FIG. 6 is a drawing showing an embodiment of the invention where a catheter is connected to a pump by a sliding type connector; 1

FIGS. 7A-C is a drawing showing an embodiment of the invention where a catheter is connected to a pump by a sliding type connector (variation of embodiment shown in FIG. 6)

FIG. 8 is a drawing showing a cross section of a catheter connected to a pump by a connector according an embodiment in FIG. 7;

FIG. 9 is a drawing showing a cross section of a catheter connected to a pump by a connector according an embodiment in FIG. 7; and

FIG. 10 is a drawing showing an embodiment of the invention where a stopping mechanism is affixed to a catheter to prevent a connector ring from sliding off the catheter.

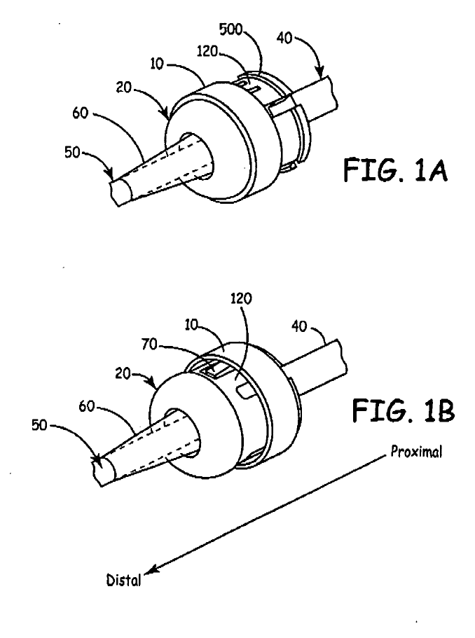

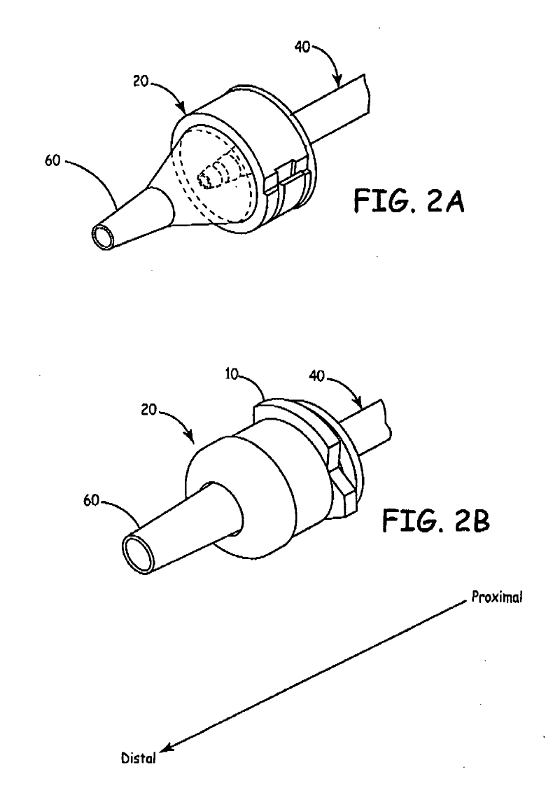

The drawings are not necessarily to scale. Like numbers refer to like parts or steps throughout the drawings. However, it should be understood that use of like reference numbers are for convenience and should not be construed as limiting. For example, the use of the number “10” to refer to “lock” in both FIGS. 1 and 2 does not indicate that the lock of FIG. 1 must take the form of the lock shown in FIG. 2, as is clearly depicted in the figures.

### DETAILED DESCRIPTION

[20] In the following descriptions, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense.

I. Overview

A. Connector

An embodiment of the invention provides a connector for securing a catheter 50 to an implantable pump 300. The connector comprises a restraint 20 configured to secure an implantable pump 300 relative to a catheter 50. The connector further comprises a lock 10 configured to secure the restraint 20 in a secured position relative to the pump 300 and the catheter 50.

In an embodiment, the restraint 20 is configured to engage a portion of the pump 300, such as a port 40 or portion thereof, upon compression, in a radially inward direction, of at least a portion of the restraint 20. For example, the restraint 20 may comprise an extension member 120 comprising a proximal portion and a distal portion and configured to secure the catheter 50 to the implantable pump 300. At least a portion of the extension member 120 is compressible in a radially inward direction, and at least a portion of the proximal portion of the extension member 120 may be configured to engage the pump 300 when the extension member 120 is compressed. For example, the extension member 120 may comprise a feature 90 complementary to a pump feature 80, and the extension member feature 90 may grippingly engage the pump feature 80 when the extension member 120 is compressed. The extension member feature 90 and the complementary pump feature 80 may be shaped in any manner such that the extension member feature 90 engages the pump feature 80 to secure the restraint 20 to the pump 300. For example, the pump feature 80 may comprise an indentation, such as a groove, and the extension member feature 90 may comprise a protrusion, or the extension member feature 90 may comprise an indentation and the pump feature 80 may comprise a protrusion. The lock 10 may be configured to compress the extension member 120 of the restraint 20 to secure the extension member 120 of the restraint 20 to the implantable pump 300. The extension member 120 may be expandable. In an embodiment, the extension member 120 is expandable such that upon relief of a compressing force the proximal portion of the extension member is capable of being expanded substantially to its pre-compressed dimensions. The extension member 120 may be self-expandable or may be expanded by applying a radially outward force to the extension member 120, preferably to the proximal portion of the extension member 120.

In an embodiment, the restraint 20 is configured to engage a portion of the pump 300, such as a port 40 or portion thereof, in a relaxed state. The restraint 20 may comprise an extension member 120 configured to engage the pump 300, and at least a portion of the extension member 120 may be extendable in a radially outward direction. The restraint 20 and its extension member 120 preferably have sufficient resiliency to return to substantially the pre-expanded dimensions upon release of a radially outward expanding force. The extension member 120 may comprise a feature 90 complementary to a feature 80 of a portion of the pump 300 to which the restraint 20 engages, and the extension member feature 90 may be configured to grippingly engage the pump feature 80. The extension member feature 90 and the complementary pump feature 80 may be shaped in any manner such that the extension member feature 90 engages the pump feature 80 to secure the restraint 20 to the pump 300. For example, the pump feature 80 may comprise an indentation and the extension member feature 90 may comprise a protrusion, or the extension member feature 90 may comprise an indentation and the pump feature 80 may comprise a protrusion. The lock 10 is configured to prevent the restraint 20 from disengaging the pump 300.

In an embodiment, the connector comprises a tab 70 configured to secure the lock 10 in a position about the restraint 20. The restraint 20 may comprise the tab. Preferably, a distal portion of the restraint 20 comprises the tab 70. The distal portion of the restraint 20 comprising the tab 70 may be compressible in a radially inward direction, allowing the lock 10 to be disposed about the restraint 20 removed from about the restraint 20. The distal portion of the restraint 20 comprising the tab 70 is preferably expandable, so that the tab 70 may secure the lock 10 in a position relative to the restraint 20. The distal portion of the restraint 20 comprising the tab 70 is preferably expandable such that upon relief of a radially inward compressing force the distal portion of the restraint 20 comprising the tab 70 is capable of being expanded to substantially to its pre-compressed dimensions. In an embodiment, the distal portion of the restraint 20 comprising the tab 70 is self-expandable. In a preferred embodiment, the lock 10 is configured to slide over the tab 70, in e.g. a linear or helical manner, compressing the distal portion of the restraint 20 comprising the tab 70 as the lock 10 slides over the tab 70. The distal portion of the restraint 20 comprising the tab 70 is configured to expand substantially to its pre-compressed dimensions after the lock 10 is slid over the tab 70, such that the tab 70 secures the lock 10 in a position about the restraint 20. The size and shape of the tab 70 can be a compromise between ease of use and potential damage to the catheter 50. Of course, the lock 10 may comprise an expandable portion configured to expand in a radially outward direction upon sliding over the tab 70. If the lock 10 comprises an expandable portion, the expandable portion of the lock 10 is preferably contractible such that contraction of the expandable portion of the lock 10 prevents the lock 10 from sliding over the tab 70, thereby securing the lock 10 a position about the restraint 20. Of course, a distal portion of the restraint 20 comprises the tab 70 may be compressible and portion of the lock 10 may be expandable.

In addition to engaging the pump 300, the connector engages a catheter 50 to secure the catheter 50 in a position relative the pump 300. The connector may engage the catheter 50 via the restraint 20. For example, the restraint 20 may comprise a mating element 140 configured to engage a complementary element 150 of the catheter 50. The restraint mating element 140 and the complementary catheter element 150 may be shaped in any manner such that the restraint mating element 140 engages the catheter element 150 to secure the restraint 20 to the catheter 50. For example, the restraint mating element 140 may comprise an indentation and the complementary catheter element 150 may comprise a protrusion, or the restraint mating element 140 may comprise a protrusion and the complementary catheter element 150 may comprise an indentation. A portion of the catheter 50 may be tapered and may be configured to engage the lock 10 at location of the tapered portion having an outer diameter substantially similar to an inner diameter of the lock 10.

In an embodiment, the restraint 20 and the lock 10 are a single piece. In an embodiment, the restraint 20 and the lock 10 are separate pieces and may individually comprise one or more components.

A connector according to various embodiments of the invention may be secured to a pump 300 or a catheter 50 through any means. For example, a physician may secure a connector to a pump 300 by hand. Tools may also be used as deemed necessary by the physician during the connection process. Any tool the physician deems suitable may be used. Preferably, tools are not needed to connect the catheters 50 and connectors to the pump 300. If tools are needed, the tools are preferably standard tools that can be found in operating rooms.

Any biocompatible and biostable material, or combinations thereof, capable of performing the desired securing function may be used to make a connector or components thereof. Nonlimiting examples of suitable material include titanium, polycarbonate, polysulfonate, nylon, polyurethane, polyether ether ketone and polypropylene, P1700 available from Solvay (BP Amoco) Polymers Europe and Gharda is an example of a suitable polysulfonate.

An example of a suitable nylon is grilamid nylon 12, TR 55 available from EMS-Grivory. An example of a suitable polyurethane is polyurethane 75D, Tecothane TT-1075DM available from Thermedics division of Viasys Healthcare. The lock 10 and restraint 20 may be made of the same or different material. For example, the lock may be made of titanium and the restraint may be made of polysulfonate.

In an embodiment, the restraint 20 and the lock 10 of a connector are different colors. Different colored restraints 20 and locks 10 may be desirable. For example, when a restraint 20 comprises a tab 70 to maintain the lock in position, the tab 70 will be more readably identifiable when the tab 70 is a different color than the lock 10. If the tab 70 is releasable, the different colored (relative to the lock 10) tab 70 can be readily identified to facilitate disconnecting catheter 50 from a pump 300.

Any coloring agent may be used to color lock 10 and a restraint 20. Preferably, coloring agents are biocompatible. The duration of biocompatible coloring agents should be considered. For example, some coloring agents are currently deemed suitable for short-term implant. Some are suitable for long-term implant. Examples of biocompatible coloring agents suitable for long term implant include TiO₂ and carbon black.

A connector according to an embodiment of the invention may be compatible with Medtronic's model SynchroMed EL, SynchroMed II, and/or IsoMed pumps 300. Preferably a connector resists accidental disconnection of a catheter 50 from a pump 300 after implantation. Further, it is preferred that a connector is readily disconnectable after implantation, even after fibrosis.

B. Catheter

In embodiment, the invention provides a catheter 50. The catheter 50 comprises a tubular member 170 comprising a distal portion and a proximal portion. The proximal portion is configured for connection to an implantable infusion pump 300. The catheter may further comprise a seal 60. The seal 60 may be molded to the catheter 50. The seal 60 may be made of any material suitable for forming a fluid barrier. For example, the seal may be made of silicone. The seal 60 is preferably disposed about the proximal portion of the tubular member 170. The seal comprises a seal element 150. The seal element 150 may be a catheter element 150 as described herein. The catheter further comprises a restraint 20 disposed about the seal 60 and configured to engage a portion of an implantable infusion pump 300 to secure the catheter 50 to the pump 300. The restraint 20 may be part of a connector as described herein. The restraint 20 may be a restraint 20 as described herein and comprises a mating element 140 configured to engage the seal element 150. The catheter 50 may further comprise a lock 10 as described herein. The catheter may further comprise a lock stopping mechanism 100 to prevent the lock 10 from sliding off the catheter 50. The lock stopping mechanism 100 is preferably located distally to the restraint 20.

C. System

In an embodiment, the invention provides a system. The system comprises a catheter 50 having a proximal end and a distal end. The system further comprises an implantable infusion pump 300. The system additionally comprises a connector. The connector comprises a restraint 20 configured to secure the proximal end of the catheter 50 relative to the implantable pump 300. The connector further comprises a lock 10 configured to secure the restraint 20 in a position relative to the pump 300 and the catheter 50. The connector, catheter 50, restraint 20, and lock 10 may be any connector, catheter 50, restraint 20, and lock 10 as described herein. The pump 300 may comprise a port to which the catheter 50 may be secured via the connector. The port 40 may comprise a port feature 80. The port feature 80 may be a pimp feature 80 as described herein. The restraint 20 may comprise a feature 90 complementary to the port feature 80, wherein the restraint feature 90 is configured to engage the port feature 80 to secure the restraint 20 to the pump 300.

D. Methods

Various methods for securing a catheter 50 to an implantable infusion pump 300 are contemplated. Based on the disclosure herein, many such methods will be readily evident to one skilled in the art. By way of example, an embodiment of the invention provides a method for securing a catheter 50 to an implantable infusion pump 300. The method comprises inserting a portion of a port 40 of the pump 300 into the catheter 50. The method further comprises positioning a restraint 20 about the port 40 and the catheter 50 such that the restraint 20 engages the port 40 and the catheter 50. The method further comprises sliding a lock 10 over the restraint 20 to secure the restraint 20 in a position relative to the pump 300 and the catheter 50.

Various methods for releasing a catheter 50 securely coupled to an implantable infusion pump 300 are contemplated. Based on the disclosure herein, many such methods will be readily evident to one skilled in the art. By way of example, an embodiment, the invention provides a method for releasing a catheter 50 from an implantable pump 300. The method comprises depressing a tab 70 on a restraint 20 that is securely coupling the catheter 50 relative to the implantable pump 300. The method further comprises sliding a lock 10 over the depressed tab 70 to remove the lock 10 from the restraint 20. The method further comprises uncoupling the catheter 50 from the pump 300.

II. FIGS.

Referring to FIG. 1, a catheter 50 connected to a port 40 of a pump 300 by a sliding type ring connector is shown. The connector comprises a restraint 20 and a lock. The restraint 20 has a proximal portion and a distal portion. The proximal portion of the restraint comprises a plurality of extension members 120. A proximal portion of extension member 120 is configured to engage the port 40. The restraint 20 may be slid about the catheter 50. The lock 10 may be slid about the restraint 20. Sliding the lock 10 from a distal to proximal direction about the restraint 20 may result in the lock 10 sliding over tab 70. The tab 70 may be compressed radially inward as lock 10 slides over tab 70. Alternatively, the lock 10 may be expanded radially outward as lock 10 is slid over the tab 70. After the lock 10 is slid over the tab 70, the tab 70 expands or the lock 10 contracts such that the lock 10 is held in a longitudinal position relative to the restraint 20 by the tab 70. Of course there may be a plurality of tabs 70. The lock 10 ensures that the restraint 20 remains in a substantially fixed longitudinal position relative to port 40. The restraint 20 comprises a flange 500 to ensure the lock 10 remains in a locked position. The tabs 70 may be depressed to allow the lock 10 to be removed from the restraint 20. The flange 500 has an outer diameter greater than the diameter of an opening on the proximal portion of the lock 10.

The catheter 50 in FIG. 1 comprises a seal 60. The seal 60 is disposed about the proximal portion of the catheter 50. The seal 60 is tapered, having its smallest diameter distally in the taper and its largest diameter proximally in the taper. The distal portion of the restraint 20 comprises an opening having a diameter. The restraint 20 may be slid from a distal to proximal direction over the catheter 50 until the opening of the restraint 20 engages a portion of the taper of the seal 60 having a substantially similar diameter of the opening. Locking the restraint 20 in a position about the port 40, thereby secures the catheter 50 relative to the pump 300.

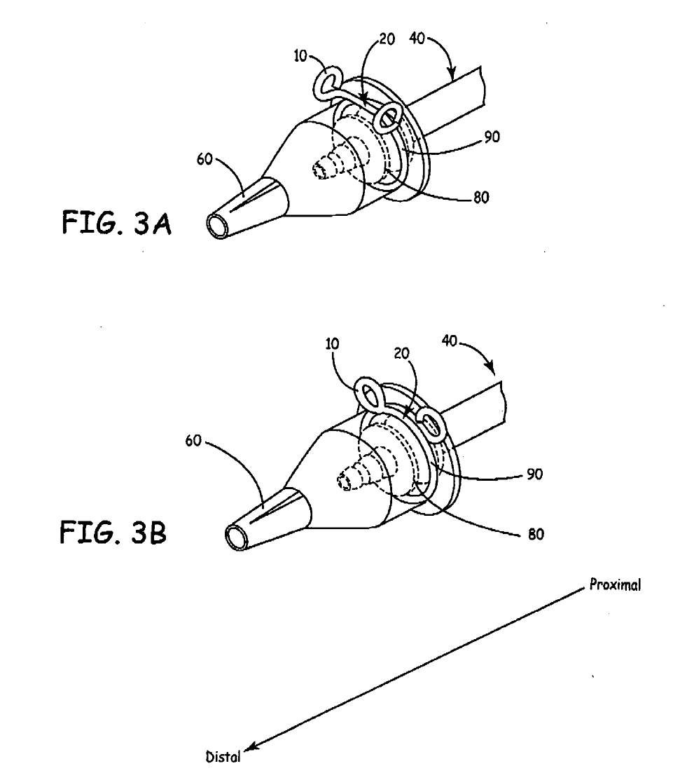

FIG. 2 shows a seal 60 of a catheter 50 connected to a port 40 of a pump 300 by a snap type connector. FIG. 3 shows a seal 60 of a catheter 50 connected to a port 40 of a pump 300 by a snap rings type connector. FIGS. 2A and 3A show an unlocked position and FIGS. 2B and 3B show a locked position.

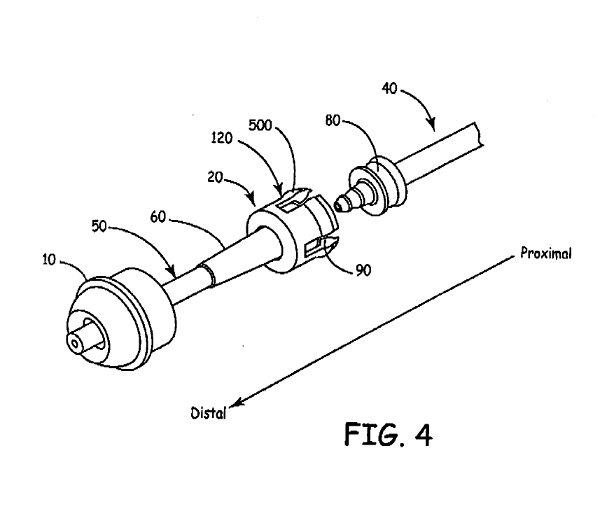

Referring to FIG. 4, an embodiment of a sliding ring type connector, a catheter 50, and a system of the invention is shown. The catheter 50 comprises a tapered seal 60 as discussed for FIG. 1. The restraint 20 comprises an opening through its distal end. The opening has a diameter similar to a portion of the tapered seal 70. The proximal portion of the restraint 20 comprises a plurality of extension members 120 configured to engage a port 40 of a pump 300. The port 40 comprises a feature 80, shown as a groove in FIG. 4. The extension members 120 comprises a complementary feature 90, shown as protrusions. A feature 90 (protrusion) of the extension member 120 is configured to engage the feature 80 (groove) of the port 40. The feature 90 (protrusion) of the extension member 120 may engage the feature 80 (groove) of the port 40 when compressed or in its relaxed state.

The lock 10 shown in FIG. 4 comprises a distal opening having a diameter. The diameter of the distal opening of the lock 10 is less than the outer diameter of flanges 500 of extension members 120 of restraint 20, such that when lock 10 is slid over restraint 20 into a locked position the lock 10 does not slide off restraint 20 proximally.

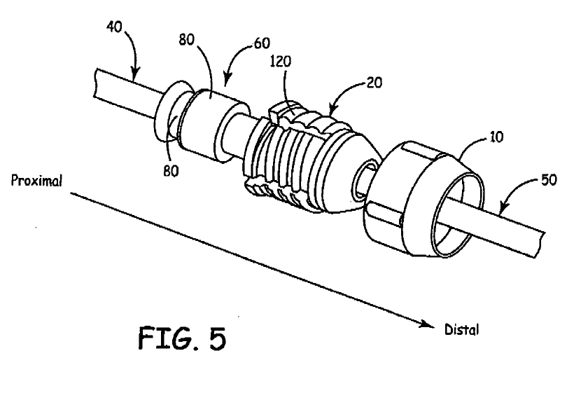

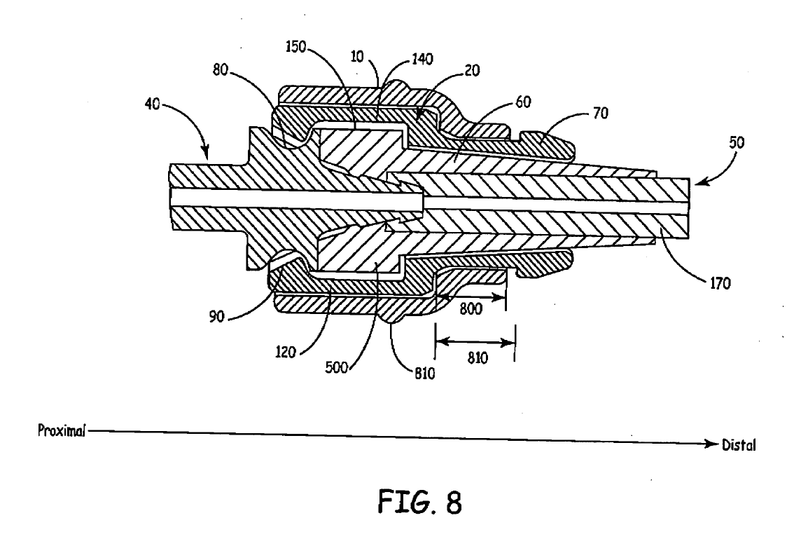

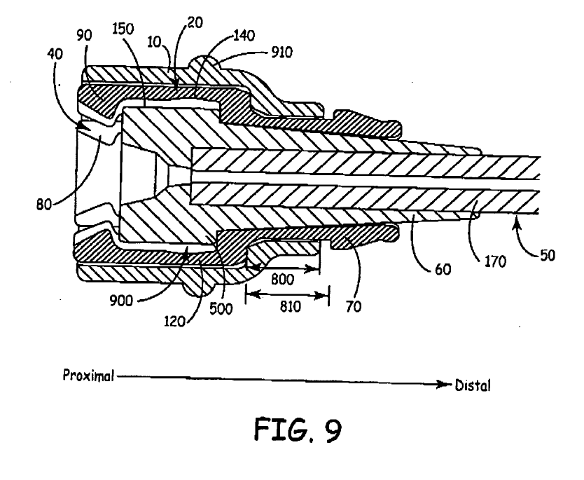

Referring to FIG. 5, an embodiment of a screw on lock type connector, a catheter 50, and a system of the invention is shown. The restraint 20 shown in FIG. 5 comprises a plurality of grooves on its exterior surface. The lock 10 comprises complementary protrusions (not shown) allowing lock 10 to be screwed into a secured position about restraint 10. Otherwise, the connector of FIG. 5 may be used to secure catheter 50 to port 40 in a similar manner to the connectors of FIGS. 1 and 4. Alternatively, as shown in FIG. 5, the seal 60 comprises a feature 80, allowing for the connector of FIG. 5 to be used to secure catheter 50 to port 40 in a similar manner to the connectors of FIGS. 8 and 9.

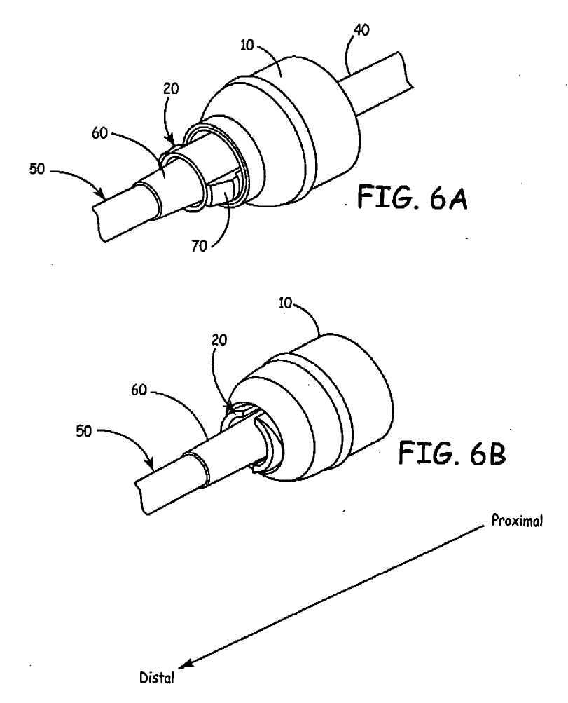

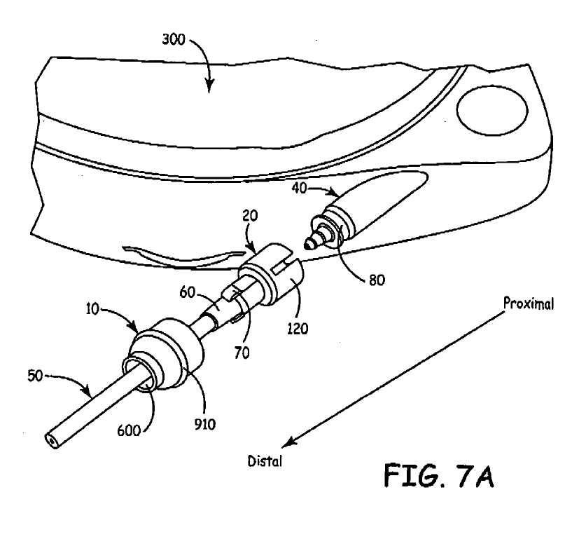

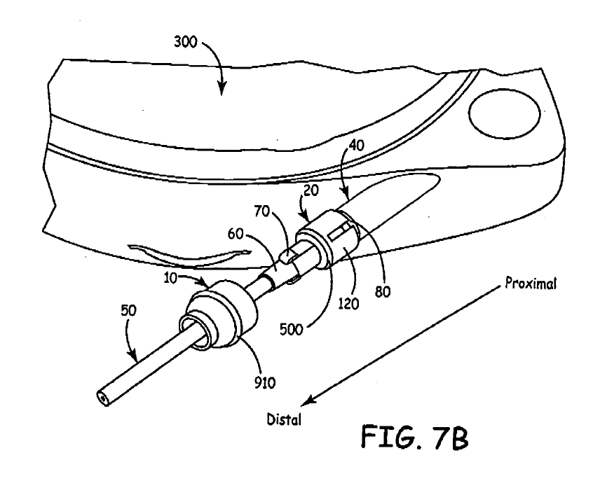

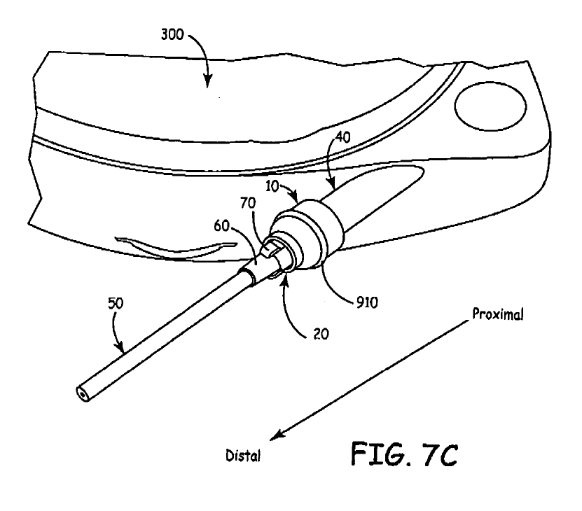

Referring to FIGS. 6-10, a catheter 50 connected to a port 40 of an implantable pump 300 by a sliding type connector is shown. The connector comprises a restraint 20 configured to secure the catheter 50 relative to the implantable pump 300. The connector further comprises a lock 10 configured to secure the restraint 20 in a secured position relative to the pump 300 and the catheter 50. The restraint 20 is comprises a plurality of extension members 120. Extension member 120 comprises a feature 90 configured to engage a complementary feature 80 of port 40. Extension member feature 90 may engage complementary feature 80 of port in extension member's 120 relaxed state or upon compression. Lock 10 may compress extension member 120 such that feature 90 of engages port feature 80. If extension member 120 engages port 40 in a relaxed state, extension member 120 may be expanded in a radially outward direction and then un-expanded to facilitate placement of extension member 120 about port 40 such that extension member feature 90 will engage port feature 80.

Restraint in FIGS. 6-10 engages catheter 50. Catheter 50 comprises a tapered seal 60, like the catheter described in FIG. 1. Restraint 20 comprises an opening towards or at its distal end. The restraint distal opening has a diameter similar to a portion of the tapered portion of the seal 60, where restraint 20 may engage seal 60. Seal 60 also comprises an element 150 and restraint 20 comprises a complementary mating element 140 configured to engage element 150 to further facilitate retaining connector in a longitudinal direction relative to catheter 50. Complementary mating element 140 may comprise an undercut.

Lock 10 in FIGS. 6-10, comprises an opening 600 having a diameter smaller than flange 500 of restraint 20, preventing lock from moving too far proximally relative to the restraint. Opening 600 of lock 10 has a diameter less than the outer diameter formed by tabs 70, such that after lock is slid over tabs 70, tabs 70 prevent lock 10 from sliding off restraint 20 in a distal direction. A portion of the lock 10 near opening 600 having a smaller diameter than flange 500 has a length 800. The length 800 of this portion of the lock 10 is less than or equal to a length 810 between tab 70 and flange 500 of restraint, thereby allowing the restraint 20 to retain the lock 10 in a defined range of longitudinal positions relative to restraint 20. Lock 10 may also comprise a protrusion 910 for ergonomic purposes to facilitate sliding lock 10 into position about restraint 20.

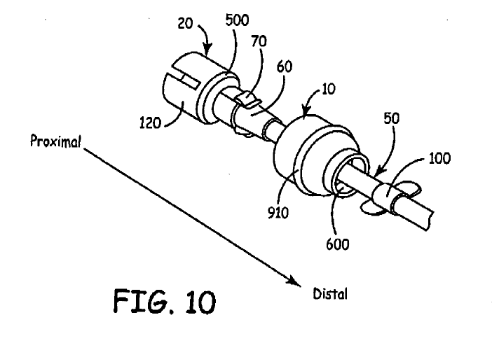

FIG. 10 shows a catheter 50 comprising a lock stopping mechanism 100. The lock stopping mechanism 100 is configured to prevent the lock 10 or restraint 20 from sliding off the distal end of catheter 50. The lock stopping mechanism 100 or a portions thereof have a diameter greater than the distal opening of the restraint and the opening 600 of the lock.

Various embodiments of the invention are disclosed. One skilled in the art will appreciate that the present invention can be practiced with embodiments other than those disclosed.

The disclosed embodiments are presented for purposes of illustration and not limitation, and the present invention is limited only by the claims that follow.

1. A connector for securing a catheter (50) to an implantable pump (300), the connector comprising:
a restraint (20) configured to secure an implantable pump (300) relative to a catheter (50); and
a lock (10) configured to secure the restraint (20) in a secured position relative to the pump (300) and the catheter (50).

2. The connector of claim 1, wherein the restraint (20) comprises an extension member (120) configured to secure the catheter (50) to the implantable pump (300).

3. The connector of claim 2, wherein the extension member (120) comprises a proximal portion and a distal portion, wherein at least a portion of the extension member (120) is compressible in a radially inward direction, and wherein at least a portion of the proximal portion is configured engage the pump (300) when the extension member (120) is compressed.

4. The connector of claim 3, wherein the extension member (120) comprises an extension member feature (90) complementary to a pump feature (80), the extension member feature (90) being configured to grippingly engage the pump feature (80) when the extension member (120) is compressed.

5. The connector of claim 4, wherein the pump feature (80) comprises an indentation and the extension member feature (90) comprises a protrusion.

6. The connector of claim 4, wherein the extension member feature (90) comprises an indentation and the pump feature (80) comprises a protrusion.

7. The connector of claim 3, wherein the lock is configured to compress the extension member (120) of the restraint (20) to secure the extension member (120) of the restraint (20) to the implantable pump (300).

8. The connector of claim 3, wherein the extension member (120) is expandable.

9. The connector of claim 8, wherein the extension member (120) is expandable such that upon relief of a compressing force the proximal portion of the extension member (120) is capable of being expanded substantially to its pre-compressed dimensions.

10. The connector of claim 8, wherein the extension member (120) is self-expandable.

11. The connector of claim 1, wherein the restraint (20), in a relaxed state, is configured to engage the implantable pump (300).

12. The connector of claim 11, wherein the restraint (20) comprises an extension member (120) configured to engage the pump (300), at least a portion of the extension member (120) being extendable in a radially outward direction.

13. The connector of claim 12, wherein the extension member (120) comprises a complementary feature (90) to a feature (80) of a portion of the pump to which the restraint (20) engages, the complementary feature (90) being configured to grippingly engage the feature (80).

14. The connector of claim 13, wherein the feature (80) comprises an indentation and the complementary feature (90) comprises a protrusion.

15. The connector of claim 13, wherein the complementary feature (90) comprises an indentation and the feature (80) comprises a protrusion.

16. The connector of claim 11, wherein the lock (10) is configured to prevent the restraint (20) from disengaging the pump (300).

17. The connector of claim 1, wherein the restraint (20) and the lock (10) are a single piece.

18. The connector of claim 1, wherein the restraint (20) and the lock (10) are separate pieces.

19. The connector of claim 1, wherein the restraint (20) further comprises a tab (70) configured to secure the lock (10) in a position about the restraint (20).

20. The connector of claim 19, wherein a distal portion of the restraint (20) comprises the tab (70).

21. The connector of claim 20, wherein the distal portion of the restraint (20) comprising the tab (70) is compressible in a radially inward direction.

22. The connector of claim 21, wherein the distal portion of the restraint (20) comprising the tab (70) is expandable.

23. The connector of claim 22, wherein the distal portion of the restraint (20) comprising the tab (70) is expandable such that upon relief of a compressing force the distal portion of the restraint (20) comprising the tab (70) is capable of being expanded to substantially to its pre-compressed dimensions.

24. The connector of claim 22, wherein the distal portion of the restraint (20) comprising the tab (70) is self-expandable.

25. The connector of claim 23, wherein the lock (10) is configured to slide over the tab (70), compressing the distal portion of the restraint (20) comprising the tab (70) as the lock (10) slides over the tab (70), and the distal portion of the restraint (20) comprising the tab (70) is configured to expand substantially to its pre-compressed dimensions after the lock (10) is slid over the tab (70), the tab (70) thereby securing the lock (10) in the position about the restraint (20).

26. The connector of claim 19, wherein the lock (10) comprises an expandable portion configured to expand upon sliding over the tab (70).

27. The connector of claim 26, wherein the expandable portion of the lock (10) is contractible, wherein contraction of the expandable portion of the lock (10) prevents the lock (10) from sliding over the tab (70), thereby securing the lock (10) the position about the restraint (20).

28. The connector of claim 27, wherein a distal portion of the restraint (20) comprises the tab (70), wherein the distal portion of the restraint (20) comprising the tab (70) is compressible in a radially inward direction, wherein compression of the distal portion of the restraint (20) comprising the tab (70) allows the lock (10) to slide over the tab (70) from the position on the restraint (20).

29. The connector of claim 1, wherein the restraint (20) comprises restraint mating element (140) configured to engage a complementary catheter element (150).

30. The connector of claim 29, wherein the restraint mating element (140) comprises an indentation and the complementary catheter element (150) comprises a protrusion.

31. The connector of claim 29, wherein the restraint mating element (140) comprises a protrusion and the complementary catheter element (150) comprises an indentation.

32. The connector of claim 3, wherein the restraint (20) comprises restraint mating element (140) configured to engage a complementary catheter element (150).

33. The connector of claim 8, wherein the restraint (20) comprises restraint mating element (140) configured to engage a complementary catheter element (150).

34. The connector of claim 12, wherein the restraint (20) comprises restraint mating element (140) configured to engage a complementary catheter element (150).

35. A catheter (50) comprising:
a tubular member (170) comprising a distal portion and a proximal portion, the proximal portion being configured for connection to an implantable infusion pump (300);
a seal (60) disposed about the proximal portion of the tubular member and comprising a seal element;
a restraint (20) disposed about the seal (60) and configured to engage a portion of an implantable infusion pump (300) to secure the catheter (50) to the pump (300), the restraint (20) comprising a mating element (140) configured to engage the seal element (150), the restraint mating element (140) being complementary to the seal element (150).

36. The catheter (50) of claim 35, wherein the restraint (20) comprises a restraint feature (90) configured to engage a complementary feature (80) of the implantable infusion pump (300).

37. The catheter (50) of claim 35, further comprising a lock (10) configured to secure the restraint (20) in a position relative to the pump (300) and the catheter (50).

38. The catheter (50) of claim 37, wherein the restraint (20) further comprises a tab (70) configured to secure the lock in a position about the restraint (20).

39. The catheter (50) of claim 38, wherein a distal portion of the restraint (20) comprises the tab (70).

40. The catheter (50) of claim 39, wherein the distal portion of the restraint (20) comprising the tab (70) is compressible in a radially inward direction.

41. The catheter (50) of claim 37, wherein the restraint (20) and the lock (10) are a single piece.

42. The catheter (50) of claim 37, wherein the restraint (20) and the lock (10) are separate pieces.

43. The catheter (50) of claim 37, further comprising a lock stopping mechanism (l 00) to prevent the lock (10) from sliding off the distal end of the catheter (50).

44. The catheter (50) of claim 43, wherein the lock stopping mechanism (100) is located distally to the restraint (20).

45. A system comprising:
a catheter (50) having a proximal end and a distal end;
an implantable infusion pump (300); and
a connector comprising
a restraint (20) configured to secure the implantable pump (300) relative to the proximal end of the catheter (50); and
a lock (10) configured to secure the restraint (20) in a position relative to the pump (300) and the catheter (50).

46. The system of claim 45, wherein the pump (300) comprises a port (40) to which the catheter (50) is secured via the connector.

47. The system of claim 46, wherein the port (40) comprises a port feature (80) and the restraint (20) comprises a restraint feature (90) complementary to the port feature (80), the restraint feature (90) being configured to engage the port feature (80) to secure the restraint (20) to the pump (300).

48. The system of claim 47, wherein the restraint (20) comprises an extension member 120, wherein the extension member 120 comprises a proximal portion and a distal portion, wherein the proximal portion comprises the restraint feature (90), and wherein the restraint feature (90) engages the port feature (80) when the proximal portion is compressed.

49. The system of claim 48, wherein the proximal portion of the extension member 120 is compressible in a radially inward direction.

50. The system of claim 48, wherein the lock (10) is configured to compress the proximal portion of the extension member (120) of the restraint (20) to secure the restraint (20) to the implantable pump (300).

51. The system of claim 48, wherein the proximal portion of the extension member (120) is expandable.

52. The system of claim 51, wherein the proximal portion of the extension member (120) is expandable such that upon relief of a compressing force the proximal portion of the extension member (120) is capable of being expanded substantially to its pre-compressed dimensions.

53. The system of claim 51, wherein the proximal portion of the extension member (120) is self-expandable.

54. The system of claim 46, wherein the restraint (20), in a relaxed state, is configured to engage the port (40).

55. The system of claim 54, wherein the restraint (20) comprises an extension member (120) configured to engage the port (40), the extension member (120) being extendable in a radially outward direction.

56. The system of claim 55, wherein the extension member (120) comprises a complementary feature (90) to a feature (80) of the port (40), the complementary feature (90) being configured to grippingly engage the feature (80).

57. The system of claim 55, wherein the lock (10) is configured to prevent the restraint (20) from disengaging the pump (300).

58. The system of claim 45, wherein the restraint (20) and the lock (10) are a single piece.

59. The system of claim 45, wherein the restraint (20) and the lock (10) are separate pieces.

60. The system of claim 45, wherein the restraint (20) further comprises a tab (70) configured to secure the lock (10) in a position about the restraint (20).

61. The system of claim 60, wherein a distal portion of the restraint (20) comprises the tab (70).

62. The system of claim 61, wherein the distal portion of the restraint (20) comprising the tab (70) is compressible in a radially inward direction.

63. The system of claim 62, wherein the lock (10) is configured to slide over the tab (70), compressing the distal portion of the restraint (20) comprising the tab (70) as the lock (10) slides over the tab (70), and the distal portion of the restraint (20) comprising the tab (70) is configured to expand substantially to its pre-compressed dimensions after the lock (10) is slid over the tab (70), the tab (70) thereby securing the lock (10) in the position about the restraint (20).

64. The system of claim 61, wherein the lock (10) comprises an expandable portion configured to expand upon sliding over the tab (70).

65. The system of claim 64, wherein the expandable portion of the lock (10) is contractible, wherein contraction of the expandable portion of the lock (10) prevents the lock (10) from sliding over the tab (70), thereby securing the lock (10) in the position about the restraint (20).

66. The system of claim 45, wherein the restraint (20) comprises a mating element (140) configured to engage a complementary element (150) of the catheter (50).

67. The system of claim 45, wherein the proximal end of the catheter (50) comprises a seal (60) and the restraint (20) is configured to engage the seal (60) to secure the catheter (50) relative to the pump (300).

68. The system of claim 67, wherein the seal (60) comprises silicone.

69. The system of claim 67, wherein the restraint (20) comprises a mating element (140) and the seal (60) comprises a complementary element (150), the restraint mating element (140) configured to engage the seal complementary element (150).

70. The system of claim 45, wherein the catheter (50) is configured to receive a portion of the port (40).

71. The system of claim 45, wherein the catheter (50) comprises a lock stopping mechanism (100) to prevent the lock (10) from sliding off the distal end of the catheter (50).

72. The system of claim 71, wherein the lock stopping mechanism (100) is located distally to the restraint (20).

73. A method for securing a catheter (50) to an implantable infusion pump (300), the method comprising:
inserting a portion of a port (40) of the pump (300) into the catheter (50);
positioning a restraint (20) about the port (40) and the catheter (50) such that the restraint (20) engages the port (40) and the catheter (50); and
sliding a lock (10) over the restraint (20) to secure the restraint (20) in a position relative to the pump (300) and the catheter (50).

74. A method for releasing a catheter (50) from an implantable pump (300), the method comprising:
depressing a tab (70) on a restraint (20), the restraint (20) securely coupling the catheter (50) relative to the implantable pump (300);
sliding a lock (10) over the depressed tab (70) to remove the lock (10) from the restraint (20); and
uncoupling the catheter (50) from the pump (300).
